(19) United States
(12) Patent Application Publication
(10) Pub. No.: US 2006/0127342 A1
(43) Pub. Date: Jun. 15, 2006

(54) Taurine-based compositions, therapeutic methods, and assays

(76) Inventor: Georgia Levis, New York, NY (US)

Correspondence Address: GIFFORD, KRASS, GROH, SPRINKLE & CITKOWSKI, P.C, PO BOX 7021, TROY, MI, 48007-7021, US

(21) Appl. No.: 11/296,112

(22) Filed: Dec. 7, 2005

Related U.S. Application Data
(60) Provisional application No. 60/634,626, filed on Dec. 9, 2004.

(51) Int. Cl. A61K 8/46; A61K 31/59; A61K 31/573; A61K 31/195

(52) U.S. Cl. 424/70.21; 514/171; 514/167; 514/567; 424/59; 514/269

(57) ABSTRACT

Taurine and derivatives thereof may be used to repair the skin barrier, enhance hydration of the skin, prevent apoptosis and oxidative damage in the skin, and heal or prevent photo-induced skin damage. The presence of biogenic taurine or taurine derivatives in tissues and/or body fluids can be used as a biomarker for tissue trauma. Detection and/or quantification of such biogenic taurine or derivatives form the basis for an assay for determining the presence and/or extent of tissue damage.

### RELATED APPLICATION

This application claims priority of U.S. Provisional Patent Application Ser. No. 60/634,626 filed Dec. 9, 2004, entitled “Taurine-Based Compositions, Therapeutic Methods, and Assays.”

### FIELD OF THE INVENTION

This invention relates generally to the diagnosis and treatment of diseases. More specifically, the invention relates to compositions and methods for the treatment and prevention of dermatological diseases, based upon the use of taurine and its derivatives. The invention further relates to the use of taurine as a biomarker for tissue stress and damage.

### BACKGROUND OF THE INVENTION

Taurine is a biomolecule which contains a sulfonate group and an amine group. As such, taurine is broadly classified as an amino acid even though it does not include a carboxylate function. Taurine is not incorporated into protein, but it is present in high concentrations in mammalian plasma and cells and plays an important role in a number of essential biological processes such as the development of the central nervous system and the retina, calcium modulation, membrane stabilization, reproduction and immunity. Owing to relatively low levels of the enzyme cysteinesulfinic acid decarboxylase, the level of biosynthesis of taurine in humans is low. Taurine occurs naturally in animal-derived foods, particularly meats and seafood.

Taurine has previously been used in various therapies and utilitarian compositions. For example, nutritional supplements often include taurine. Taurine is known to function as a chelating agent for divalent ions such as calcium, and as such has been utilized for that purpose in various topical compositions. For example, U.S. Pat. No. 5,866,142 discloses the use of taurine as a chelating agent for calcium ions in an exfoliating composition. U.S. Pat. No. 5,869,068 similarly relies upon the ionic interactions of taurine to provide a composition in which the taurine acts as an agonist for chloride ion transport channels in cells, and thereby produces a slackening of the skin which reduces the appearance of wrinkles. U.S. Pat. No. 6,562,802 discloses compositions in which taurine is combined with other chelating molecules such as chitosan to promote wound healing. Taurine has also been reacted with silicone polymers to produce a material having a surfactant property, and U.S. Pat. Nos. 5,280,099 and 5,286,830 disclose such compositions and their various uses. While taurine has been noted to have some physiological effects, both internally and topically, its full range of utility has not been heretofore understood or exploited.

In accord with the present invention, it has been found that topical application of taurine-containing compositions is of benefit in a number of dermatological conditions. Furthermore, it has been found that taurine is a very effective biomarker for tissue trauma; hence, measurement of taurine levels in tissues or body fluids can be used to determine existence and amount of tissue trauma an organism has experienced. These and other aspects of the present invention will be described in detail hereinbelow.

### SUMMARY OF THE PRESENT INVENTION

Disclosed herein is a method for enhancing the hydration of the skin. The method comprises applying to the skin a composition comprising taurine or a derivative thereof. In particular embodiments, the derivative of taurine is selected from the group consisting of salts, esters, complexes and conjugates of taurine. In a specific instance, the derivative of taurine comprises a chloramine/taurine complex. The composition may further include active ingredients such as retinoids, emollients, topical anesthetics, moisturizers, corticosteroids, permeation enhancers, sunscreens, antibiotics, coloring agents, fragrances and combinations thereof. In other instances, the composition may also include vitamin D, analogues of vitamin D, calcineurin inhibitors, an immunomodulating drug, an anticancer agent, 5-FU, diclofenac and combinations thereof. The compositions may also include a vehicle comprising materials such as physiological lipids, non-physiological lipids, phospholipids, triglycerides, diglycerides, monoglycerides, free fatty acids, fatty alcohols, ceramides, cholesterol, cholesterol esters, isoflavonoids and various combinations thereof. In specific instances, the concentration of taurine in the composition is in the range of 1-20 weight percent.

Also disclosed is a method for repairing the skin barrier. The method comprises applying to the skin a topical composition which comprises taurine or a derivative thereof. Further ingredients as discussed above may also be included in the composition.

In yet another embodiment of the present invention, there is disclosed a method for preventing photo damage to the skin by applying a composition which inhibits apoptosis in the skin. The active agent for inhibiting apoptosis may comprise taurine or a derivative thereof. The composition may further include an ultraviolet light absorbing sunscreen material.

Also disclosed are methods for inhibiting aging of the skin by applying a composition which comprises taurine or a derivative thereof.

Further disclosed is a method for treating diseases associated with the nails by applying a composition which includes taurine or a derivative thereof to the nails. Further disclosed is a method for detecting and/or quantifying trauma to the tissue of a patient's body. The method comprises the step of assaying a tissue or body fluid of the patient to determine the presence and/or quantity of taurine or its derivatives in the tissue or fluid. Further disclosed are specific compositions for practicing the methods of the present invention.

### DETAILED DESCRIPTION OF THE INVENTION

In accord with the first aspect of this invention, it has been found that the topical application of taurine and its derivatives is of significant benefit in treating a number of dermatological conditions. It is significant that taurine is a material which carries the designation “generally regarded as safe.” Within the context of this patent “taurine” is understood to also include derivatives of taurine such as its salts and esters as well as its complexes, conjugates and the like; and specifically includes biogenic taurine derivatives such as chloramine/taurine complexes, as well as synthetic and semi-synthetic derivatives. Within the context of this patent, all references to “taurine” are understood to also include derivatives of taurine.

One embodiment of the taurine-based therapy of the present invention involves repair of the skin barrier. The epidermis forms a barrier between the moisture-rich tissues of an individual's body and the relatively dry exterior environment. The outermost layer of the skin, the stratum corneum which is largely composed of corneocytes derived from epidermal keratinocytes, is constantly exposed to water loss placing it under persistent osmotic stress. Taurine is found in the epidermis of humans and a number of other animal species, and it is believed that the dermal cells counter water loss by increasing the amount of taurine and other such osmolytes therein. It has also been found that biogenic taurine is actively transported into the epidermis where it is believed to function to protect cells from dehydration, and it has been determined that taurine accumulation in the skin is increased under induced osmotic stress.

In view of the foregoing, the present invention provides for enhanced skin barrier protection by the use of externally applied taurine supplements. As such, taurine functions as a humectant which increases the hydration of dehydrated, or potentially dehydrated, skin so as to enhance the integrity of the skin barrier. In accord with the present invention, there are provided topical compositions of taurine in a carrier vehicle, and these compositions function to restore the integrity of the skin barrier, rehydrate the skin and prevent moisture loss.

The concentration of taurine used in various preparations of the present invention will vary depending upon the specific application, the type of carrier vehicle, and other such factors. However, in one group of embodiments, the concentration of taurine in the topical preparation is in the general range of 1-20 weight percent. One particular group of compositions has a concentration in the range of 5-15 percent; another group of compositions includes approximately 1-5 percent by weight of taurine. In specific compositions, concentrations of approximately 2.5 percent by weight are employed.

The carrier vehicle for the various compositions of the present invention may comprise an aqueous, lotion, gel, or a lipid-rich emollient vehicle as is well known in the art. In one group of embodiments, the carrier is a liposomal, or other lamellar or multi-lamellar, vehicle. It is to be understood that ancillary ingredients may be included in the compositions of the present invention. These ancillary ingredients can include dermally active drugs such as vitamin D and its analogues, calcineurin inhibitors and other immunomodulating drugs, anticancer agents and other drugs indicated for actinic keratosis, including, but not limited to, 5-FU, diclofenac and the like, as well as other substances known to repair and maintain the skin barrier function. Such substances include, but are not limited to: physiological lipids, such as essential and non-essential fatty acids, phospholipids, ceramides, cholesterol and its esters, isoflavonoids and other protease inhibitors. In the instance where skin barrier repair is contemplated, these ancillary ingredients can also include active ingredients such as retinoids, corticosteroids, antibiotics and the like. The ancillary ingredients may also include emollients, topical anesthetics, permeation enhancers, sunscreens, colors, fragrances and other such materials as is known in the art.

In accord with another aspect of the present invention, it has been found that taurine can protect normal human epidermal keratinocytes from ultraviolet induced cell death (apoptosis), even at micromolar concentrations. This effect is independent from and in addition to, the skin barrier repair and hydrating effects taurine exerts on dermal cells.

In accord with this particular aspect of the present invention, taurine-based compositions of the type described hereinabove are effective for both preventing and healing photo-induced skin damage. Given the fact that taurine itself is not a very strong absorber of those ultraviolet wavelengths which contribute to skin tanning, compositions of taurine may be used to permit tanning while preventing various types of skin damage. In other instances, taurine compositions may include ultraviolet absorbing sun block agents of the type known in the art. Also, as described above, the compositions may include further therapeutic and non-therapeutic materials.

Taurine's actions in repairing the skin barrier, restoring hydration to the skin, preventing apoptosis, and preventing oxidative damage to tissue will cause it to have utility in compositions for preventing or alleviating chronological or photo aging of the skin. In this regard, taurine may be incorporated into cosmetics as well as therapeutic preparations such as lotions and the like.

It is to be understood that within the context of this disclosure, skin is understood to include mucosal tissues. The compositions of the present invention will have utility for hydrating and restoring mucosal tissues. For example, the taurine-based compositions may be used on buccal or vaginal tissues, and will also have utility in the ano-rectal region for treating inflammations, hemorrhoids, fissures, and other conditions.

Because of the presence of the sulfonate moiety, taurine and its derivatives can interact with the keratinous matrix of the nails so as to enhance their permeability and facilitate the transport of materials into and through the nails. Furthermore, taurine and various of its derivatives have a direct antimicrobial effect. Hence, taurine, used either alone or with other therapeutic materials such as antifungal drugs, can be effective in treating onychomycosis, psoriatic nail disorders, and other diseases associated with the nails.

While the foregoing has primarily described compositions based upon the free amino acid taurine, it is to be understood that derivates of taurine may likewise be employed in the practice of the present invention. For example, taurine may be utilized in the form of a salt or an ester. Taurine may also be employed in the form of various conjugates, and given the chemical reactivity of the sulfonate and amine group, a number of such conjugates will be readily apparent to those of skill in the art. For example, taurolidine is a taurine-based conjugate based upon taurine and formaldehyde. This material is known in the art, and has been used as an antibacterial material, and may be utilized in the practice of various aspects of the present invention either with or without free taurine. Chloramine/taurine complexes may be similarly employed.

In view of the teaching presented herein, one of skill in the art could readily formulate various topical compositions of taurine. One specific composition comprises, on a weight basis:

| | |
|---|---|
| Taurine | 2.5% |
| Cetostearyl alcohol | 7.2% |
| Ceteth-20 | 1.8% |
| Light mineral oil | 6% |
| White petrolatum | 15% |
| Propyl paraben | 0.15% |
| Trolamine (99%) | 1% |
| Triethanolamine lauryl sulfate | 0.25% |
| Purified water | 66.6% |

The foregoing is a cream-based taurine composition. It may be used as a skin barrier repair product, moisturizer or skin protectant and can function to alleviate photo-induced skin damage, inhibit oxidative skin damage, and inhibit apoptosis. Further ingredients as noted above, as well as ancillary ingredients such as fragrances, coloring agents and the like, may be incorporated thereinto. Likewise, the composition may be formulated as a lotion, an emollient or fatty cream or ointment, gel, foam or other pharmaceutically acceptable vehicle for topical use. For example, preparations used to prevent sun damage will preferably be made as low viscosity lotions or as water-based compositions.

A composition similar to the foregoing was prepared utilizing 1.5 weight percent taurine. Both of the compositions were evaluated in a patient having both psoriasis and eczema. Both creams were found to be excellent moisturizers for xerotic skin. Neither was irritating to dry or normal skin, nor did either irritate the psoriasis or eczema. Both the psoriasis and eczema improved within the 1.5% and 2.5% creams, thereby demonstrating that in addition to the skin barrier repair and moisturizing effect, taurine has anti-inflammatory qualities.

In accord with yet another aspect of the present invention, it has been found that taurine levels in various tissues increase as a result of physical damage or other trauma; and biogenic taurine can be a marker for such trauma. Specifically, the detection of elevated levels of taurine or its derivatives will suggest that an organism has suffered some tissue trauma. Likewise, quantification of the level of taurine or its derivatives can be used to estimate the severity of the trauma. Taurine is very water soluble, and relatively simple assays based upon the analysis of body fluids such as saliva, urine, sweat or blood for taurine can be employed. Also, levels of taurine measured in tissue samples can provide very specific information about the physiological state of that tissue. High performance liquid chromatography (HPLC) is particularly effective for detecting and quantifying levels of taurine and its derivatives in a variety of fluids. Taurine levels may be measured by a number of other analytical techniques well known in the art including immunochemical techniques as well as more classical types of chemical reagent interactions.

The present invention provides a number of therapeutic compositions and methods based upon taurine-containing topical preparations. Also, the present invention provides for an analytical method for detecting and/or quantifying tissue trauma based upon the measurement of taurine levels. Some specific descriptions and examples have been presented herein. In view of this teaching, yet other embodiments and versions of the present invention will be readily apparent to those of skill in the art. The foregoing discussion and description is illustrative of specific embodiments of the invention, but is not meant to be a limitation upon the practice thereof. It is the following claims, including all equivalents, which define the scope of the invention.

1. A method for enhancing the hydration of the skin, said method comprising:
applying to the skin a composition comprising taurine or a derivative thereof.

2. The method of claim 1, wherein said derivative of taurine is selected from the group consisting of salts, esters, complexes and conjugates of taurine.

3. The method of claim 1, wherein said derivative of taurine comprises a chloramine/taurine complex.

4. The method of claim 1, wherein said composition further includes a member selected from the group consisting of: retinoids, emollients, topical anesthetics, moisturizers, corticosteroids, permeation enhancers, sunscreens, antibiotics, coloring agents, fragrances and combinations thereof.

5. The method of claim 1, wherein said composition further includes a member selected from the group consisting of: vitamin D, analogues of vitamin D, calcineurin inhibitors, an immunomodulating drug, an anticancer agent, 5-FU, diclofenac and combinations thereof.

6. The method of claim 1, wherein said composition further includes a member selected from the group consisting of: physiological lipids, non-physiological lipids, phospholipids, ceramides, cholesterol, cholesterol esters, isoflavonoids, protease inhibitors, and combinations thereof.

7. The method of claim 1, wherein said vehicle includes liposomes.

8. The method of claim 1, wherein the concentration of taurine or said taurine derivative is in the range of 1-20 weight percent.

9. A method for repairing the skin barrier, said method comprising:
applying to the skin a topical composition which comprises taurine or a derivative thereof.

10. A method for preventing photo damage to the skin, said method comprising the step of:
applying to the skin a composition which inhibits apoptosis in said skin.

11. The method of claim 10, wherein said composition which inhibits apoptosis comprises taurine or a derivative thereof.

12. The method of claim 10, wherein said composition is applied to said skin in conjunction with an ultraviolet absorbing sunscreen material.

13. A method for inhibiting the aging of skin, said method comprising the step of:
applying to said skin a composition which comprises taurine or a derivative thereof.

14. A method of treating diseases associated with the nails, said method comprising applying to said nails a composition which includes taurine or a derivative thereof.

15. The method of claim 14, wherein said diseases are selected from the group consisting of: onychomycosis and psoriatic nail disorders.

16. A method for detecting and/or quantifying trauma to tissue of a patient's body, said method comprising the step of:
assaying a tissue or body fluid of said patient to determine the presence and/or quantity of taurine or its derivatives in said tissue or fluid.

17. The method of claim 16, wherein said body fluid is selected from the group consisting of blood, urine, saliva, sweat, or combinations thereof.

18. The method of claim 16, wherein said tissue comprises skin, lungs, gastrointestinal tissue, and muscle.
